(19) United States
(12) Patent Application Publication
(10) Pub. No.: US 2006/0247671 A1
(43) Pub. Date: Nov. 2, 2006

(54) Compact, multi-use micro-sampling device

(76) Inventor: Richard W. LeVaughn, Sharpsburg, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/415,961

(22) Filed: May 2, 2006

Related U.S. Application Data
(60) Provisional application No. 60/676,914, filed on May 2, 2005.

(51) Int. Cl. A61B 17/32; A61B 5/00

(52) U.S. Cl. 606/182; 600/583

(57) ABSTRACT

A compact, multi-use lancing device includes a lancet holder for holding a lancet and mounted for back and forth movement along a lancing stroke path within a housing, and a drive mechanism having a pair of generally parallel springs coupled to the lancet holder. A drive spring propels the lancet holder in a forward direction to prick the skin of a subject, and a return spring retracts the lancet holder to return the lancet to a protected position within the housing.

### CROSS-REFERENCE TO RELATED APPLICATION

This application claims the priority benefit of U.S. Provisional Patent Application Ser. No. 60/676,914 filed in the United States Patent and Trademark Office on May 2, 2005, which is hereby incorporated herein by reference in its entirety for all purposes.

### TECHNICAL FIELD

The present invention relates generally to medical devices and procedures, and more particularly to a multi-use micro-sampling device having a compact drive mechanism configuration.

### BACKGROUND OF THE INVENTION

Many medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics and in blood typing and screening applications.

In some instances, a person must periodically sample their blood for multiple testing throughout the day or week. Thus, due to the nature of micro-sampling for blood glucose monitoring, many individuals requiring regular monitoring prefer to do so in as discrete a manner as possible. Many users prefer a more compact lancing device for ease of carrying, storage and use, and for more discrete sampling.

Current multi-use micro-sampling devices tend to have a relatively large, elongate pen-like form that may restrict the ability of the individual user to conduct micro-sampling discretely. This elongate form of many current multi-use micro-sampling devices is commonly the result of an internal drive mechanism configuration wherein a drive spring and a return spring (typically in the form of coil springs) operate in series on a lancet carrier, meaning that the drive and return springs are typically aligned coaxially in-line with one another. For example, a drive spring and a return spring may be coaxially mounted at the rear and front ends, respectively, of a linear cylindrical lancet carrier component. Each of these springs has a length sufficient to generate the desired path and velocity profile of the lancet's lancing stroke. Because the springs are positioned coaxially in-line with one another, their cumulative lengths plus the length of the lancet carrier or a portion thereof, typically result in a relatively elongate drive mechanism, which in turn necessitates a relatively long housing and considerable overall device length.

Previous efforts to design a more compact, multi-use micro-sampling device have typically focused on shortening the individual components of a drive system having its drive and a retraction springs in series, to make the overall length of the device shorter. In such designs, stronger springs are typically required to create equivalent energy with shorter deflection. Thus, the user tends to experience a higher force to load the potential energy into the system. Also, shorter springs, requiring smaller deflection, tend to not be very precise in their tolerances. And the velocity profile of the lancing stroke typically cannot be as precisely controlled with shorter, stiffer springs, often leading to increased sensation of pain by the subject, which may result in decreased compliance with a prescribed sampling regimen.

Previously known multi-use micro-sampling device drive mechanism configurations have also been found disadvantageous, in that their drive and/or return springs generally must be held or placed in a state of compression and/or tension during assembly of the device. This can increase the complexity and cost of the assembly process, often leading to a more expensive overall device.

Thus it can be seen that needs exist for improvements to multi-use micro-sampling lancing devices to provide a more compact device, allow more discrete sampling, and facilitate easier assembly. Needs further exist for such a mechanism that has a smaller overall profile while remaining compatible with standard lancets commonly available. In addition, needs exist for combining drive elements for control of the lancet travel and return that provide for a shorter overall configuration, but still maintain a desired velocity profile along the lancing stroke. Needs also exist for integrating a lancing device into a testing meter, such as a glucose meter, to reduce the amount of equipment persons need to carry.

### SUMMARY OF THE INVENTION

Briefly described in example forms, the present invention is a multi-use micro-sampling or lancing device comprising a compact outer housing containing drive and activation mechanisms for receiving a disposable and replaceable lancet, and driving the lancet through a controlled lancing stroke for sample collection. The housing preferably includes a lancet holder or carrier that securely but releasably engages the lancet and constrains the lancet along a controlled and pre-defined path of travel during the lancing stroke.

Example forms of the lancing device preferably include a drive mechanism including two or more parallel springs working to drive and propel the lancet through an extended position along its lancing stroke, and to return the lancet to a retracted position fully within the housing. This drive mechanism is relatively compact, and thus the overall size, and particularly the length, of the lancing device can be significantly reduced. In this manner, example embodiments of the lancing device of the present invention enable more discrete sampling by users, and/or allow the lancing device to more readily be integrated into a test meter or other device to reduce the number of testing items a user must carry. Example forms of the lancing device of the present invention also enable assembly without the need for holding and/or placing the drive and/or return spring(s) in a state of compression or tension, advantageously facilitating more economical manufacture.

In one aspect, the invention is a lancing device including a compact housing; a lancet carrier for a holding a lancet and mounted for movement along a lancing stroke path within the housing; and a drive mechanism having at least two springs coupled in parallel to the lancet carrier, wherein a first spring of the at least two springs drives the lancet carrier in a forward direction and a second spring of the at least two springs retracts the lancet carrier.

In another aspect, the invention is a lancing device including a drive mechanism having at least two springs, and a lancet carrier having at least two elongate shafts extending parallel to one another. Each of the springs is preferably mounted over a corresponding shaft of the lancet carrier.

In another aspect, the invention is a lancing device including a drive spring, a return spring, and a lancet carrier upon which the drive spring and return spring are mounted along generally parallel axes.

And in still another aspect, the invention is a lancing device including a housing; a lancet carrier translationally mounted within the housing for engaging a lancet and carrying the lancet along a path of travel; a drive spring operable on the lancet carrier; and a return spring operable on the lancet carrier, and wherein the return spring has a return spring axis parallel to the path of travel of the lancet and parallel to a drive spring axis of the drive spring.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is an assembly view of a lancing device according to an example embodiment of the present invention.

FIG. 2 is a perspective view of the drive mechanism components of the lancing device shown in FIG. 1.

FIG. 3 is a sectional view of the multi-use lancing device of FIG. 1 with its drive mechanism shown in an “equilibrium” or “free” state.

FIG. 4 is a sectional view of the lancing device of FIG. 1 with its drive mechanism shown in a “charged” or “energized” state.

FIG. 5 is a top sectional view of the lancing device of FIG. 1 with its drive mechanism shown in an “extended” or “lancing” state.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, as for example by use of the antecedent “about,” it will be understood that the particular value forms another embodiment.

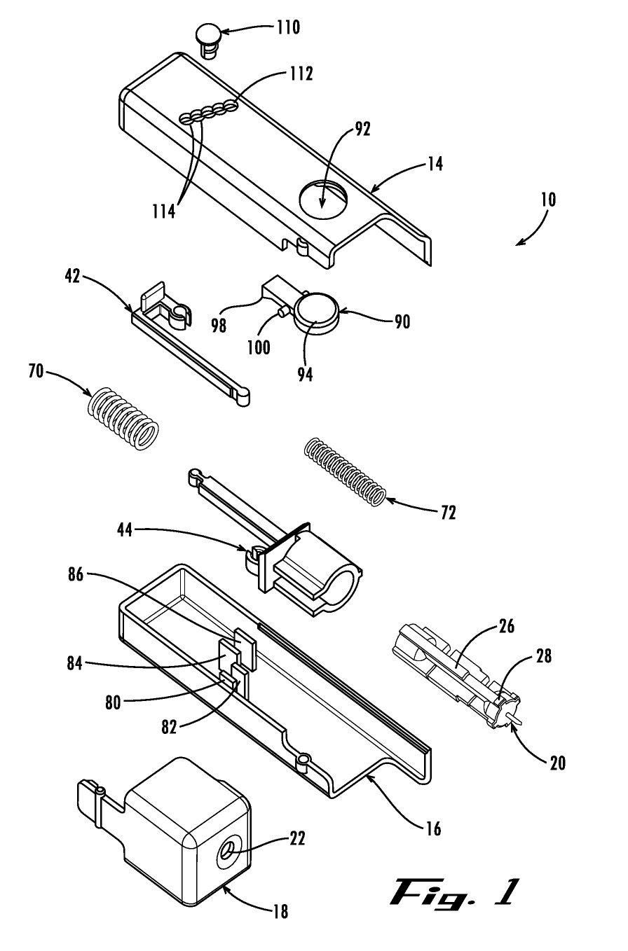

With reference now to the drawing figures, FIG. 1 is an assembly view of a multi-use micro-sampling device or lancing device 10 according to an example embodiment of the present invention. The lancing device 10 preferably comprises a compact housing 12 having a top portion 14 connected to a bottom portion 16, as for example by adhesive, snap-fittings, fastener(s) or crush-pins. At one end of the device 10 is a hinged or removable endcap 18 of the lancing device 10 that can be opened such that a used lancet 20 can be manually removed from the device and a new lancet can be manually inserted into the device. The endcap 18 preferably defines an opening 22 for protrusion of a sharp lancet needle or blade tip 24 into the tissue at the intended lancing site selected by user.

A replaceable and disposable lancet 20 is preferably installed in a reciprocating lancet holder, carrier or sled 40 translationally mounted within the housing 12 for reciprocating movement along a lancing stroke or path of travel. The lancet 20 typically comprises a needle or blade forming a sharp lancet tip 24 and a lancet body 26, and is positioned such that the tip 24 is directed forward, toward the lancet opening 22 in the endcap 18. Preferably, the lancet body 26 is formed of plastic, and is injection-molded around the needle or blade 24. The lancet tip 24 is preferably encapsulated by a protective sterility cap 28, which is integrally molded with the lancet body 26 and forms a sterility and safety barrier for the lancet tip. Preferably, the protective sterility cap 28 of each lancet 20 is releasably connected to the lancet body 26 at a transition region, which is an area of weakening such that it is easily detached and removed from the lancet body 26. The lancet 20 can be, for example, any of a variety of standard, commercially-available lancets.

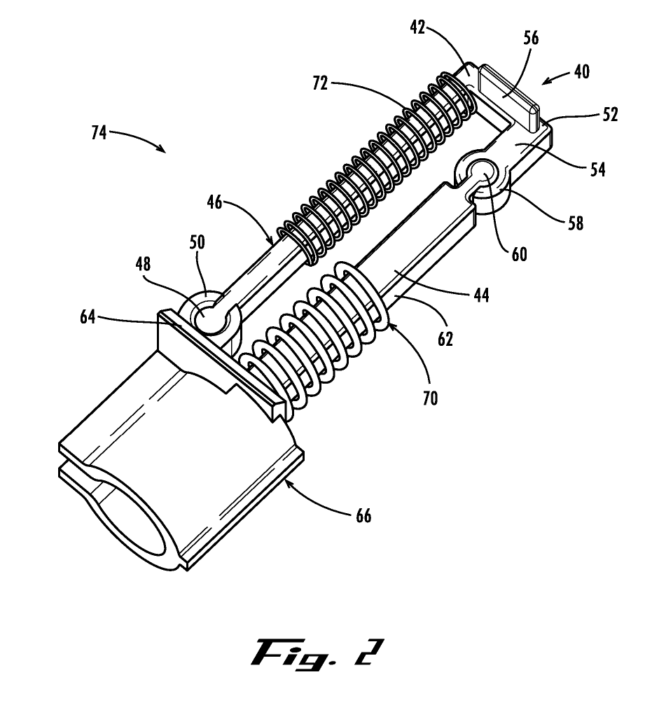

As more clearly seen in FIG. 2, the lancet carrier 40 preferably comprises a first portion 42 and a second portion 44. The first portion 42 resembles an L or U-shaped crook having a shaft or post portion 46 with a male connector 48 at its distal end for mating with a cooperating female connector 50 of the second portion 44 of the carrier 40; a transverse member 52; and a hook 54. The transverse member 52 preferably has a fin or tongue 56 extending therealong in the direction towards the upper portion of the housing when the sled 40 is mounted therein, for cooperative contact with a depth adjuster in the top portion of the housing. The hook 54 preferably has a female connector 58 for mating with a cooperating male connector 60 of the second portion 44 of the lancet carrier 40. In alternate embodiments, the male and female connectors of the first and second portions of the carrier are reversed.

The second portion 44 of the lancet carrier 40 preferably includes a shaft or post portion 62 having the male connector 60 at its distal end, for mating engagement with the cooperating female connector 58 of the first portion of the carrier. The post portion 62 and the female connector 50 are secured or affixed to, and are preferably integral with, an upper surface of a panel or shelf portion 64 of the carrier 40. A sleeve or yoke 66 preferably extends from the lower surface of the panel or shelf portion 64 for securely but releasably holding a lancet 20 therein. The first portion 42 and the second portion 44 preferably mate together to form an open, generally rectangular lancet carrier assembly having sides formed by the shafts 46, 62, and ends formed by the transverse member 52 and the panel 64.

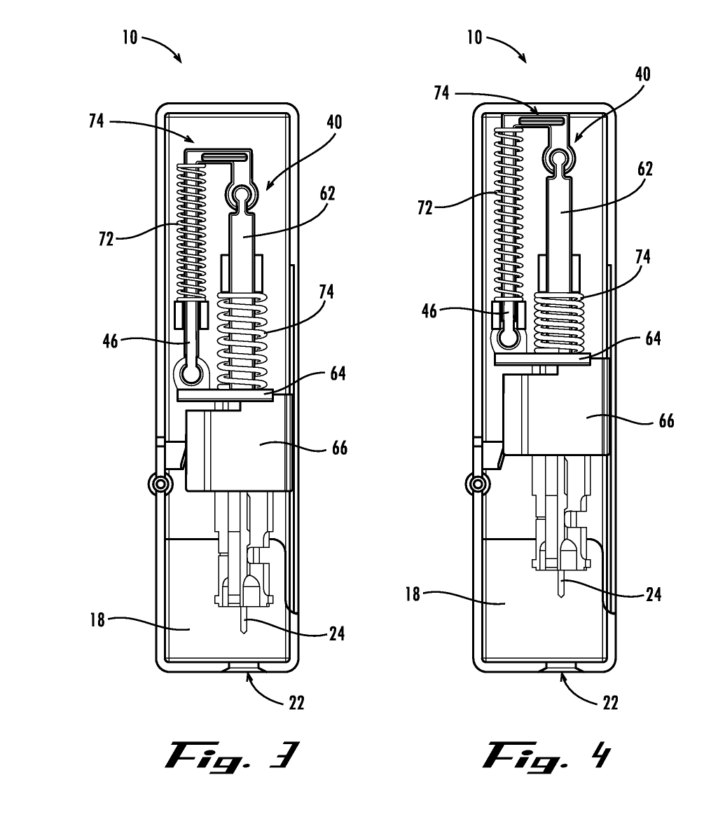

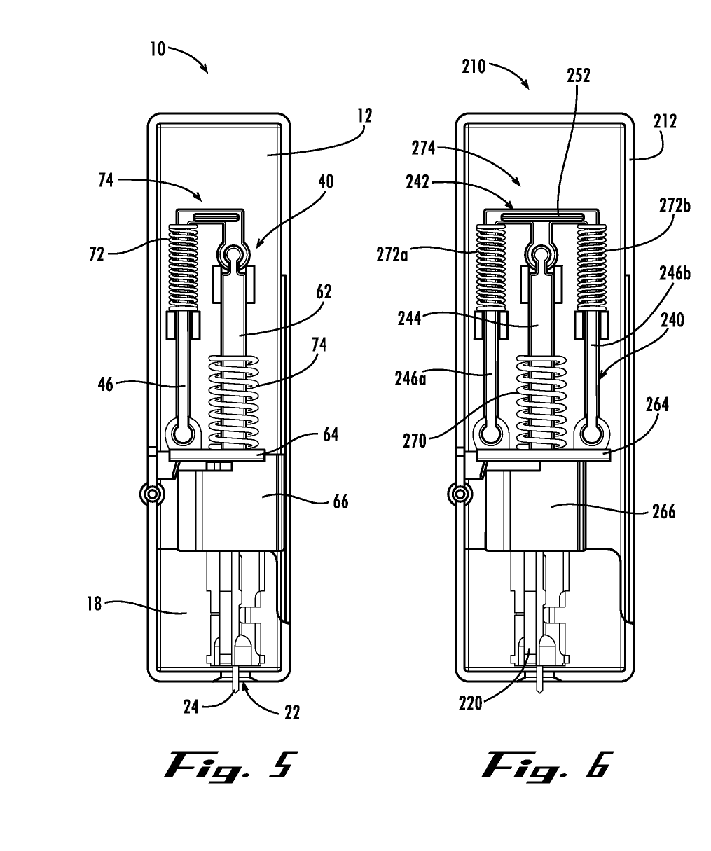

Two springs, namely a drive spring 70 and a return spring 72, preferably operate in tandem to drive and return the lancet carrier 40 along a desired lancing stroke or path of travel upon energizing and activating of the lancing device 10. Together, the drive spring 70, the return spring 72, and the lancet carrier 40 form a drive mechanism 74 for driving the lancet 20 back and forth along its pre-defined path. The drive spring 70 is preferably the stronger of the two springs, and drives the lancet 20 from its retracted or energized position (FIG. 4) into its extended position (FIG. 5). The return spring 72 serves to retract the lancet 20 back into the equilibrium position (FIG. 3) within the housing 12 after lancing the skin.

With reference to FIG. 1 and FIG. 2, in the depicted embodiment, the return spring 72 is preferably mounted onto and around the first shaft 46, and the drive spring 70 is preferably mounted onto and around the second shaft 62. In alternate embodiments, the spring configuration may be the reverse. The mating connectors of the first and second carrier components 42, 44 allow the springs to be installed onto the carrier 40 prior to attachment of the first and second carrier components to one another, without holding or placing the springs in compression or tension. Upon engagement of the connectors of the first and second carrier components 42, 44 together, the springs 70 and 72 are held and constrained on the side shafts of the carrier 40 and are positioned generally parallel to (i.e., not co-axially in-line with) one another. This construction allows for installation of the springs 70 and 72 in a single subassembly before it placement into the lancing device 10, as well as providing for a relatively compact arrangement of the drive mechanism 74.

The lower portion 16 of the housing 12 preferably has a plurality of retaining walls or guide members projecting therefrom, for guiding the lancet carrier 40 along a predefined linear path or stroke. Preferably, there are four such retaining walls 80, 82, 84, and 86. The retaining walls 80 and 82 preferably serve to guide the first post 46 along a predefined path as well as to provide a seat for the retraction spring 72. Thus, the retraction spring 72 abuts the retaining walls 80 and 82 and acts in compression on the transverse member 52, to bias the carrier 40 towards the rear (i.e., away from the lancet opening 22) of the housing 12. Similarly, the retaining walls 84 and 86 serve to guide the second post 62 along a predefined path as well as to provide a seat for the drive spring 70. Thus, the drive spring 70 abuts the retaining walls 84 and 86 and acts in compression on the panel or shelf 64, to bias the carrier 40 forward towards the lancet opening 22.

The lancing device 10 preferably further comprises an activation mechanism or trigger release 90 for triggering the drive mechanism 74 to propel the lancet 20 through its lancing stroke. An example configuration of the activation mechanism or trigger release 90 is shown in FIG. 1, and preferably includes a button 94 at an end of a lever 96, the button positioned within an opening 92 of the top portion 14 of the housing 12. On the side of the lever 96 opposite the button 94 is a barb 98 that engages the shelf 64 of the carrier 40 when the carrier is in a “charged” or “activated” position, to hold the carrier in the charged position. When a user pushes the button 94 inwardly, the lever 96 pivots about its fulcrum 100 and releases the barb 98 from engagement with the shelf 64, which in turn releases the carrier from its charged position. In such instance, the drive spring 70 exerts a force on the carrier 40 and drives the carrier toward the front of the device (i.e., towards the opening 22) such that the lancet tip 24 protrudes through the opening 22 and punctures the skin of the user.

The lancing device 10 preferably also comprises a depth adjustment mechanism 110. The depth adjustment mechanism 110 preferably comprises a tab or pin that slides within a diagonal or curved slot 112 located in the upper portion 14 of the housing 12. The slot 112 preferably has a plurality of detents 114 therein for retaining the depth adjuster tab in one of a plurality of predetermined indexed positions. An internal end of the tab 110 contacts the tongue 56 of the carrier and acts as a mechanical stop for limiting the travel the carrier at the forward end of the lancet stroke. Thus, the user can preset how deep the lancet will penetrate the skin at the lancing site based on the position of the depth adjuster tab 110. For example, the further towards the rear of the device (i.e., the further away from the opening 22) the tab is, the less the tip of the lancet protrudes through the housing in its extended position. Conversely, the closer the tab is towards the front of the device (i.e., towards the endcap 18), the more the tip of the lancet protrudes through the housing in its extended position. Preferably, the tongue 56 is broad/long enough to contact the depth adjuster tab 110 in all of its positions. Also preferably, the slot 112 is elongated either diagonally or vertically in a direction along the length of the device 10.

FIG. 3 shows a top sectional view of the multi-use lancing device 10 with the lancet 20 inserted therein. The lancet holder 40 is not yet “cocked” or “charged”, and thus the lancet holder is considered to be in a “withdrawn”, or “steady” state. In this “withdrawn” state, the springs 70 and 72 are in their equilibrium states such that the drive spring 70 and return spring 72 counter-balance one another, and the lancet carrier 40 is in an equilibrium position. The retraction spring 72 is in a retracted compression state, or its “equilibrium” or “steady” state, and opposes the “equilibrium”, “free”, or “steady” position of the drive spring 70.

When the device 10 is in this “equilibrium” state, a user preferably manually removes any used lancet remaining in the device, loads a new lancet 20 into the lancet carrier 40 (preferably with its protective cap 28 still in place), and pushes the lancet (and thereby the lancet carrier 40) back within the housing (i.e., in the direction away from the endcap 18), until the barb 98 of the trigger mechanism 90 snaps into engagement with panel 64 of the lancet carrier, thereby placing the device in its “cocked” or “energized” state, as depicted in FIG. 4. Once the device 10 is thusly charged, the user preferably removes the protective sterility cap 28 from the lancet body 26.

In the charged drive mechanism configuration shown in FIG. 4, the drive spring 70 is under compression, and the retraction spring 72 is in its extended or relaxed state. Because the drive spring 70 is stiffer than the return spring 72, when the carrier 40 is released by actuation of the activation or trigger mechanism 90, the lancet 20 is driven forward into its extended position as depicted in FIG. 5. When the lancet 20 moves into its extended position, its sharp tip 24 protrudes through the lancing opening 22 and punctures the user's skin at a lancing site pressed against the contact face of the endcap. After lancing, the drive mechanism returns to its equilibrium or retracted position by the retraction spring 72, and the lancet is withdrawn back to a position fully within the housing 12 where the tip is shielded against inadvertent contact.

FIG. 6 shows a lancing device 210 according to another embodiment of the present invention, and having a housing 212 enclosing a reciprocating translationally mounted drive mechanism 274. The drive mechanism preferably includes a lancet carrier 240 having a split-ring sleeve or collar 266 for releasably but securely holding a disposable lancet 220. The device 210 is substantially similar in structure and operation to the above-described device 10, but includes a parallel pair of return springs 272a, 272b operating in tandem with one another and in parallel with the drive spring 270. The lancet carrier 240 preferably comprises a U-shaped first element 242 having first and second shaft or post portions 246a, 246b extending from a transverse member 252, and a second element comprising a panel 264 having the sleeve 266 extending from one side thereof and a shaft 244 extending from the other side thereof. Interengaging connectors of the first and second elements of the lancet carrier 240 are engaged with one another to complete the drive mechanism assembly after the drive spring 270 has been mounted onto the shaft 244 and the tandem return springs 272a, 272b have been mounted onto shafts 246a, 246b. The relative spring constants of the drive spring 270 and the return springs 272a, 272b are selected to drive the lancet along a desired lancing stroke, in similar fashion to that described above. Preferably, the spring constants of the return springs 272a, 272b are relatively equal, and the lancet carrier is configured to position the return springs generally parallel to and substantially equidistant from and on opposite sides of the drive spring 270, to provide a smooth and balanced drive mechanism that does not impart any significant force couple or rotation during actuation, thereby producing a more precise and linear lancing stroke path and velocity profile.

By positioning the drive and return springs of the drive mechanism in parallel with one another, rather than in series, lancing devices according to various example embodiments of the present invention can be configured to have a relatively shorter overall length, without the control difficulties resulting from the use of short, stiff springs. For example, placement of coil springs serving as the drive and return springs over spaced-apart, parallel shafts of the lancet carrier, positions the springs' axes in parallel to and optionally at least partially alongside one another, resulting in a more compact configuration than if the springs were coaxially aligned on opposite ends of the carrier. The overall length of the lancing device is preferably less than four times the length of the lancet used in connection therewith, and more preferably between about 2.5 to 3 times the length of the lancet. In a lancing device embodiment for use with a lancet having a length of about 25 mm, for example, the overall device may have a length of about 70 mm or less.

The invention also includes a method of assembly of a lancing device. The method of the invention includes providing a lancet carrier having two or more parallel shaft components, and connectors enabling drive and return springs to be mounted onto the parallel shafts of the lancet carrier. After mounting the drive and return springs onto the parallel shafts of the lancet carrier, the connectors are engaged to capture the springs and retain them in place on the carrier. The carrier is then installed into a housing with the springs preferably in a relaxed state (i.e., not in compression or tension) and between opposed surfaces or elements of the carrier and the housing.

While the invention has been described with reference to preferred and example embodiments, it will be understood by those skilled in the art that a variety of modifications, additions and deletions are within the scope of the invention, as defined by the following claims.

What is claimed is:

1. A lancing device comprising:
a compact housing;
a lancet carrier for a holding a lancet and mounted for movement along a lancing stroke path within the housing; and
a drive mechanism comprising at least two springs coupled in parallel to the lancet carrier, wherein a first spring of the at least two springs drives the lancet carrier in a forward direction and a second spring of the at least two springs retracts the lancet carrier.

2. The lancing device of claim 1, wherein the lancet carrier comprises a linkage having first and second elongate members, wherein the first elongate member carries the first spring, and the second elongate member carries the second spring.

3. The lancing device of claim 2, wherein the linkage comprises a generally open rectangular assembly having the first and second elongate members forming its sides.

4. The lancing device of claim 1, wherein the drive mechanism comprises three springs coupled in parallel to the lancet carrier, one of said three springs comprising a drive spring, and two of said three springs comprising retraction springs.

5. The lancing device of claim 1, wherein the lancet carrier comprises a first element and a second element, the first and second elements having connectors therebetween, and wherein the at least two springs are mounted onto the lancet carrier prior to engagement of the connectors.

6. A lancing device comprising a drive mechanism having at least two springs and a lancet carrier comprising at least two elongate shafts extending parallel to one another, wherein each of said springs is mounted over a corresponding shaft of the lancet carrier.

7. The lancing device of claim 6, wherein the lancet carrier comprises a first element and a second element, the first and second elements having connectors therebetween, and wherein the springs are mounted onto the shafts prior to engagement of the connectors.

8. The lancing device of claim 7, wherein the connectors comprise interengaging male and female members.

9. The lancing device of claim 6, wherein the lancet carrier comprises an open rectangular assembly with the elongate shafts forming sides thereof.

10. The lancing device of claim 6, wherein the lancet carrier has three elongate shafts extending parallel to one another, each of said three elongate shafts having a spring mounted thereon.

11. The lancing device of claim 10, wherein the three elongate shafts comprise a center shaft having a drive spring mounted thereon, and two side shafts having return springs mounted thereon.

12. The lancing device of claim 11, wherein the two side shafts are mounted on opposite sides of the center shaft from one another and approximately equidistant from the center shaft.

13. The lancing device of claim 6, wherein the device is assembled with each of the springs in a relaxed state.

14. A lancing device comprising:
a housing;
a lancet carrier translationally mounted within said housing for engaging a lancet and carrying the lancet along a path of travel;
a drive spring operable on the lancet carrier; and
a return spring operable on the lancet carrier, and wherein the return spring has a return spring axis parallel to the path of travel of the lancet and parallel to a drive spring axis of the drive spring.

15. The lancing device of claim 14, wherein the lancet carrier comprises a split collar for releasably engaging the lancet.

16. The lancing device of claim 15, wherein the lancet carrier further comprises elongate shafts upon which the drive spring and return spring are mounted.

17. The lancing device of claim 16, wherein the housing comprises guide members for engaging the elongate shafts of the lancet carrier.

18. The lancing device of claim 14, further comprising a second return spring operable on the lancet carrier, and having a second return spring axis parallel to the path of travel of the lancet.

19. A method of assembling a lancing device, comprising:
mounting a drive spring and a return spring onto parallel shafts of a lancet carrier;
engaging at least one connector to retain the drive spring and the return spring on the parallel shafts of the lancet carrier; and
installing the lancet carrier into a housing.

20. The method of claim 19, wherein the drive and return springs are in a relaxed state when the lancet carrier is installed into the housing.
